(19) United States
(12) Patent Application Publication
(10) Pub. No.: US 2021/0275446 A1
(43) Pub. Date: Sep. 9, 2021

(54) METHOD OF TREATING A VIRAL INFECTION

(71) Applicant: Morou BOUKARI, Toulouse (FR)

(72) Inventor: Morou BOUKARI, Toulouse (FR)

(21) Appl. No.: 17/248,501

(22) Filed: Jan. 27, 2021

Related U.S. Application Data
(60) Provisional application No. 63/046,157, filed on Jun. 30, 2020.
(60) Provisional application No. 62/984,588, filed on Mar. 3, 2020.
(60) Provisional application No. 63/001,759, filed on Mar. 30, 2020.

(51) Int. Cl. A61K 9/00; A61L 2/00

(52) U.S. Cl. CPC A61K 9/0043; A61L 2/0088

(57) ABSTRACT

A method that prevents or treats infection of the human body with the COVID-19 virus or any other virus by interacting with the crucial enzymes and proteins of the virus and by preventing the penetration of the virus into human cells.

### STATEMENT OF RELATED CASES

This application claims priority to U.S. provisional application Nos. 63/046,157 filed Jun. 30, 2020, 62/984,588 filed Mar. 3, 2020, and 63/001,759 filed Mar. 30, 2020. The entirety of each of these is hereby incorporated by reference.

### BRIEF SUMMARY OF THE INVENTION

Applicant's method of treating a viral infection, and an optional kit for use with the method, allows the following: Combat and/or prevent infection of the human body with the COVID-19 virus and/or any other virus; Combat and/or prevent the worsening of the infection of the human body by the COVID-19 virus and/or any other virus; Combat, and/or prevent, cytokine over-inflammation (cytokine storm) of the human body; Treat the two routes of entry of COVID-19 coronavirus into the body (nasal and oral routes); Prevent the COVID-19 virus and/or any other virus from colonizing and developing in the upper respiratory tract (nose, throat); Prevent COVID-19 virus and/or any other virus from migrating into the lower respiratory tract (lungs); Prevent the COVID-19 virus and/or any other virus from colonizing and developing in the lower respiratory tract (lungs); Prevent the virus from developing in the digestive tract and from entering the human body through the intestinal walls; Restoring energy to the human body to enable it to combat infection with the COVID-19 virus and any other virus to come.

The method is to be used for preventive and curative purposes. The method can be applied to the combat COVID-19 viruses and any other virus.

The method of prevention and treating an infection of the body with COVID-19 virus and/or any other virus is based on several technical characteristics:
Physical elimination of the COVID-19 virus and/or any other virus by physical drainage of these viruses outside of:
   Nasal passages when you blow your nose and after spraying the nasal passages with a spray containing isotonic saline or after nasal washing with isotonic saline
   The throat when spitting after gargling the throat with isotonic saline
Elimination of COVID-19 viruses and/or any other virus by activation of the body's immune defenses by supplying cells of the human body and cells located in the lower respiratory tract and in the upper respiratory tract a non-toxic chemical substance (chloride) precursor of a bactericidal and virucidal compound and obtained from isotonic saline for washing the nose or washing the throat or supplied by an appropriate diet, which substance is then transformed by cells of the human body and to the cells located in the lower respiratory tract and in the upper respiratory tract in a powerful bactericidal and virucidal compound (hypochlorous acid), and which bactericidal and virucidal compound thus obtained ensures:
   Elimination of the COVID-19 virus and/or any other virus in the lower respiratory tract and in the upper respiratory tract,
   Elimination of the COVID-19 virus and/or any other virus from the human body,
   The reduction in the production of cytokines and chemokines in the immune defense cells, thereby leading to a reduction in the inflammation of the human body.

The non-toxic chemical (chloride) precursor of bactericidal and virucidal compound can directly ensure at the level of the immune defense cells a reduction in the production of the cytokine thus leading to a reduction of the inflammation of the human body.

### DETAILED DESCRIPTION OF THE INVENTION

The method prevents infection of the human body with the COVID-19 virus by interacting with the crucial enzymes and proteins of the virus and by preventing the penetration of the virus into human cells by providing the human body with a mixture of fresh fruits naturally containing neohesperidin and hesperidin as active substances.

Neohesperidin inhibits the COVID-19 virus enzyme 3CLpro (Nsp5). This enzyme is essential for the life cycle of the COVID-19 virus. Hesperidin inhibits the COVID-19 virus Helicase protein (Nsp13). This protein is involved in the replication of the COVID-19 virus (1). Hesperidin and neohesperidin block protein S of COVID-19 virus by binding to it. This protein S allows the COVID-19 virus to penetrate human cells. Hesperidin also binds to the protein S interface of COVID-19 virus and ACE2 receptors in cells, thereby preventing the penetration of COVID-19 virus into human cells. Hesperidin and neohesperidin also bind to ACE2 receptors in human cells, preventing the COVID-19 virus from entering human cells.

The fresh fruit blend also contains vitamin C and natural sugars (an energy source for the human body) that help the human body defend itself against infection with the COVID-19 virus. This vitamin C also helps reduce the destruction of human cells by the hydrogen peroxide formed in the body during its infection by the COVID-19 virus.

The method combats the development of the COVID-19 virus and/or any other virus into the respiratory tract (lower and upper) and their penetration into the cells of the lungs thanks to the inhalation of essential oils of Scots pine, or Aleppo pine and/or using Racemic Camphor and Levomenthol.

The method prevent the development of the COVID-19 virus in the digestive and intestinal tracts and restore energy to the human body by taking an oral solution of an acid, tonic, energetic solution (between 150 and 200 KJ per 100 ml) and containing carbonated water, sugar (between 5 and 15 g per 100 ml of water), an acidifier (phosphoric acid), natural flavors and caffeine. The pH of this solution can be between 2 and 3.

A treatment kit associated with the virus comprises one or more sub-kits:

III-1) Sub-kit N º1:
A nasal spray containing a propellant gas (nitrogen) and an isotonic solution consisting of between 15 and 40% of sea water, preferably 33% of sea water, the rest (between 60 and 85%) being purified water,
Pods (5 ml each) of a physiological solution containing 0.9 g of sodium chloride per 100 ml of purified water,

1 stick containing a pad impregnated with racemic Camphor and Levomenthol,
1 vial of 10 ml containing essential oil of Scots pine
1 vial of 10 ml containing essential oil of Aleppo pine,
1 essential oil diffuser

III-2) Sub-kit N º2:
1 liter of an acidic, tonic, energetic solution (between 150 and 200 KJ per 100 ml) and containing carbonated water, sugar (between 5 and 15 g per 100 ml of water), an acidifier (phosphoric acid), natural flavors and caffeine.

III-3) Sub-kit N º3:

This sub-kit N º3 is a kit for mixing fresh fruits.
Composition: mixture composed of several varieties of fresh fruits of mandarins, clementines, oranges, grapefruit and lemons in well-defined proportions.
Example of preparation:
   Ingredients:
      Clementine
      Lime
      Bitter orange
      Grapefruit
   Preparatory mode:
      Wash all fruit in soapy water. Use the dishwashing liquid.
      Remove the skin of the clementines
      Peel the lime, removing only the outer skin
      Peel the orange, removing only the outer skin
      Cut each fruit into very small pieces so that when all these fruits are mixed, you get a homogeneous mixture and you can mask the taste of lemon and grapefruit.
   Dosage for 100 grams of mixture:
      60 grams of clementine
      20 grams of lemon
      10 grams of bitter orange
      10 grams of grapefruit
   Dosing in general:
   Dosage in mass percentage in the fresh fruit mixture:
      Clementine (all varieties): 40 to 60%
      Lemon (all varieties): 10 to 40%
      Bitter orange (all varieties): 5 to 10%
      Sweet orange (all varieties): 0 to 60%
      Grapefruit (all varieties): 0 to 10%
      If there is a shortage of clementines, all varieties of sweet oranges can be used.

In the event of a shortage, the use of bitter orange can be dispensed with.

This mixture of fresh fruits can be packaged in various forms. For example, in the form of sachets, trays, bags containing the mixture of fruits cut into small pieces to eat directly or to grind, mix and drink. For example, in the form of bottles, cans, barrels containing the mixture of fresh crushed, mixed and drinkable fruits.

III-4) Sub-kit N º4:

A tray of 0.5 to 1 kg containing salted potato puree. For this consumption, the dosage of chloride ions in the blood must be controlled. This dosage should be between 100 and 110 mmol/L (or mEq/L) and preferably around 110 mmol/L (mEq/L).

The method also comprises in certain embodiment certain precautions to take, including one or more of the following:
Wash your hands with soap and with drinking water every day before drinking, before eating, after each use of the toilet, each time you arrive at your home or place of work. If soap and water are not available, use a hydroalcoholic solution or gel.
Dry your hands preferably with disposable hand towels.
Do not leave the carafes of water in the open air.
Do not leave an opened bottle of water open to the air.
Avoid drinking not disinfected spring or well water
Disinfect door handles daily with 90-95° ethyl alcohol or using disinfecting wipes.

In a preferred embodiment, the method includes:
1—Washing of the two nostrils morning, noon and evening with:
   a. A nasal spray containing a propellant gas (nitrogen) and an isotonic solution made up between 15 and 40% of sea water, preferably 33% of sea water, the rest (between 60 and 85%) being purified water. The washing of each nostril is done by spraying the spray into each nostril for at least 10 seconds or for a period of between 5 and 20 seconds. You can use the Vitarmonyl nasal spray for example. At the end of the washing of the two nostrils, blow your nose to evacuate the nasal secretions possibly containing the viruses hung on the mucous membranes of the nostrils.
   b. Or with a physiological solution containing 0.9 g of sodium chloride per 100 ml of purified water. The washing of each nostril is done by spraying 5 ml of the physiological solution in each nostril. At the end of the washing of the two nostrils, blow your nose to evacuate the nasal secretions possibly containing the viruses hung on the mucous membranes of the nostrils.

This step 1 makes it possible to combat the penetration of COVID-19 into the human body by the nasal route. It makes it possible to remove the COVID-19 coronavirus fixed on the nasal mucous membranes.
2—After each washing of the nostrils, inhaling (2 times for each nostril) using a tampon impregnated with racemic Camphor and Levomenthol. A Vicks stick can be used for this purpose. This inhalation is to be done in the morning, at noon and in the evening and during the first 3 days of executing the method.

This step makes it possible to combat COVID-19 coronavirus fixation on the nasal mucosa and COVID-19 deep penetration into the respiratory tract.
3—Drinking once a day (well before bed) an acidic, tonic, energetic solution (between 150 and 200 KJ per 100 ml) and containing carbonated water, sugar (between 5 and 15 g per 100 ml of water), an acidifier (phosphoric acid), natural flavors and caffeine. The pH of this solution can be between 2 and 3. This tonic and energetic solution can be regular Coca-Cola or another drink equivalent to Coca-Cola. With each take, you should drink between 50 and 250 ml depending on the age of the individual. Taking this acidic, tonic and energetic solution makes it possible to combat possible diarrhea, colonization of the mucous membranes of the intestine by the virus, the penetration of this intestinal mucosa by the virus COVID-19. This tonic acid solution can be taken outside of meals or just before meals or during meals or just after meals. The total duration of taking this drink can be 2 to 3 days per week. This taking of tonic solution allows to restore energy to the body and to boost the body's defense against this COVID-19 coronavirus.
4—Diffusing in a room or a bedroom, Scots pine or Aleppo pine essential oil and breathing in this room or bedroom for a time between 10 minutes and 30 minutes. This oil helps combat the development of COVID-19 in the respiratory tract and its penetration to the lungs. This operation is to be done once a day and this for the duration of the epidemic. Inhalers can also be used by fumigation of essential oil to directly inhale this Scots pine oil. It is also possible to light a candle containing Scots pine essential oil.
5—Consuming daily the mixture of fresh fruits contained in the sub-kit N º3

Dosage:

Adult: 2 doses of 100 g each per day (morning and noon or noon and 4 p.m.).

Child: 2 doses of 50 g each per day (morning and noon or noon and 4 p.m.)

The fresh fruit mixture can be eaten directly or be mixed and taken as a drink.

In another preferred embodiment, the method includes the following precautions:
Wash your hands with soap and drinking water every day before drinking, before eating, after each use of the toilet, each time you arrive at your home or place of work. If soap and water are not available, use a hydroalcoholic solution or gel.
Dry your hands preferably with disposable hand towels.
Do not leave the carafes of water in the open air.
Do not leave an opened bottle of water open to the air.
Avoid drinking not disinfected spring or well water
Disinfect the door handles every day with ethyl alcohol 90-95° or using the disinfectant wipes.
Wear a surgical type filtering respirator mask, FFP1 or FFP2 if you cough or sneeze.

The method is to be used as a curative during the appearance of the first signs such as: stuffy or runny nose, body aches, cough, fever, bone pain, headache, diarrhea).

In another preferred embodiment, the method comprises:
1—Washing of the two nostrils morning, noon and evening with:
   a. A nasal spray containing a propellant gas (nitrogen) and an isotonic solution made up between 15 and 40% of sea water, preferably 33-35% of sea water, the rest (between 60 and 85%) being purified water. The washing of each nostril is done by spraying the spray into each nostril for at least 10 seconds or for a period of between 5 and 20 seconds. You can use the Vitarmonyl nasal spray for example. At the end of the washing of the two nostrils, blow your nose to evacuate the nasal secretions possibly containing the viruses hung on the mucous membranes of the nostrils.
   b. Or with a physiological solution containing 0.9 g of Sodium Chloride per 100 ml of purified water. The washing of each nostril is done by spraying 5 ml of the physiological solution in each nostril. At the end of the washing of the two nostrils, blow your nose to evacuate the nasal secretions possibly containing the viruses hung on the mucous membranes of the nostril.

This step 1 makes it possible to combat the penetration of COVID-19 into the human body by the nasal route. It makes it possible to unhook the COVID-19 coronavirus fixed on the nasal mucous membranes.
2—After each wash of the nostrils, performing inhalations (2 times for each nostril) using a pad impregnated with racemic Camphor and Levomenthol. A Vicks stick can be used for this purpose. These inhalations are to be performed in the morning, at midday and in the evening and during the first 3 days of treatment. This step makes it possible to combat the fixation of the COVID-19 coronavirus on the nasal mucosa and the deep penetration of COVID-19 into the respiratory tract.
3—Taking paracetamol-based drugs in the morning and in the evening for the first two days of the first signs (muscle aches, fever, bone pain, headache, diarrhea). The dose is 400 mg for an adult. This take of paracetamol helps combat fever and pain.
4—Drinking in the morning, at noon and in the evening (well before bedtime) an acid, tonic, energetic solution (between 150 and 200 KJ for 100 ml) and containing carbonated water, sugar (between 5 and 15 g for 100 ml of water), an acidifier (phosphoric acid), natural flavors and caffeine. The pH of this solution can be between 2 and 3. This tonic and energetic solution can be regular Coca-Cola or another drink equivalent to Coca-Cola or Pepsi. With each take, drink between 50 and 250 ml depending on the age of the patient. Taking this acidic, tonic and energetic solution makes it possible to combat possible diarrhea, colonization of the mucous membranes of the intestine by the virus, the penetration of this intestinal mucosa by the virus COVID-19. This tonic acid solution can be taken outside of meals or just before meals or during meals or just after meals. The total duration of taking this drink can be 3 to 5 days with a first intake at the first signs of the onset of the disease (stuffy or runny nose, muscle aches, cough, fever, bone pain, headache, diarrhea). This take of tonic solution helps restore energy to the body and boosts the body's defense against the COVID-19 coronavirus.
5—Taking paracetamol-based medicines in the morning and in the evening from the third day and for at least two days. The dose is 1 g for an adult. This take of paracetamol helps to combat aches and possible fever. Paracetamol medications can also be taken from day one, alternating with ibuprofen.
6—Diffusing Scots pine essential oil in a room or bedroom and breathe in this room or room for a time between 10 minutes and 30 minutes. This oil helps combat the development of COVID-19 in the respiratory tract and its migration to the lungs. This operation is to be done twice a day, from the first day and this for at least 5 days. Inhalers can also be used by fumigation of essential oil to directly inhale this essential oil of Scots pine or Aleppo pine. You can also light a candle containing Scots pine essential oil.
7—Consuming daily the mixture of fresh fruits contained in the sub-kit N º3

Dosage:

Adult: 2 doses of 100 g each per day (morning and noon or noon and 4 p.m.).

Child: 2 doses of 50 g each per day (morning and noon or noon and 4 p.m.)

The fresh fruit mixture can be eaten directly or mixed and taken as a drink.

1. A method of treating a viral infection of a subject in need thereof comprising:
a) washing both nostrils of the subject three times a day, once in the morning, once at midday, and once in the evening, the washing comprising:
i) spraying a nasal spray into each nostril for a period of between 5 and 20 seconds, and
ii) blowing the nose to evacuate nasal secretions containing the viruses on the mucous membranes of the subject;
b) inhaling two times for each nostril racemic camphor and levomenthol impregnated on a pad, wherein the inhaling is three times a day, once in the morning, once at midday, and once in the evening;
c) drinking between 50 and 250 ml once a day an acidic, tonic, energetic solution containing carbonated water, sugar at between 5 and 15 g per 100 ml of the water, an acidifier, natural flavors, and caffeine, wherein the pH of solution is between 2 and 3;
d) inhaling pine essential oil using an inhaler, or diffusing pine essential oil into a room and breathing in the room once a day for a time between 10 minutes and 30 minutes;
e) consuming twice per day 100 g of a mixture of fresh fruit if the subject is an adult, or 50 g per day for a child subject, the mixture comprising:
clementine, lime or lemon, bitter orange, and grapefruit mixed together,
wherein each fruit is washed in soapy water,
wherein the skin of the clementine is removed,
wherein only the outer skin of the lime and the bitter orange is removed, and
wherein each fruit is cut into pieces, and the pieces are mixed together into a homogeneous mixture of pieces and the taste of lemon and grapefruit is masked;
f) washing the subjects hands with soap and drinking water every day before drinking, before eating, after each use of the toilet, each time arriving at home or place of work, and if the soap and the drinking water is not available, washing using a hydroalcoholic solution or gel,
i) wherein after washing, drying hands with disposable hand towels;
g) avoiding drinking spring or well water that has not been disinfected, and any water left in an open carafe or open bottle exposing the water to open air;
h) disinfecting door handles in the environment of the subject daily with 90-95° ethyl alcohol or using disinfecting wipes.

2. The method of claim 1, wherein for 100 g of the mixture of fresh fruit, there is 60 grams of the clementine, 20 grams of the lime or lemon, 10 grams of the bitter orange, and 10 grams of the grapefruit.

3. The method of claim 1, wherein the mixture of fresh fruit has mass percentages of
a. clementine 40% to 60%
b. lime or lemon 10% to 40%
c. bitter orange 5% to 10%
d. sweet orange 0 to 60%
e. grapefruit 0 to 10%

4. The method of claim 1 wherein the mixture of fresh fruit is packaged in the form of sachets, trays, or bags.

5. The method of claim 1 wherein the mixture of fresh fruit is packaged in the form of bottles, cans, or barrels.

6. The method claim 1 wherein the viral infection is COVID-19.

7. The method of claim 1 wherein the acidic, tonic, energetic solution is a cola soft drink.

8. The method of claim 1 further comprising wearing a surgical type filtering respirator mask, FFP1 or FFP2 if the subject coughs or sneezes.

9. The method of claim 1 further comprising administering paracetamol-based drugs to the subject in the morning and in the evening for the first two days of the first signs of infection.

10. The method of claim 1 wherein the nasal spray comprises a propellant gas and an isotonic solution consisting of between 15% and 40% of salt water, and between 60% and 85% purified water.

11. The method of claim 10 wherein the propellant gas is nitrogen.

12. The method claim 10 wherein the salt water content of the spray is 33%.

13. The method of claim 1 wherein the nasal spray is vitarmonyl nasal spray.

14. The method of claim 1 wherein the nasal spray comprises a physiological solution containing 0.9 g of sodium chloride per 100 ml of purified water.

15. The method of claim 1 wherein the spraying is spraying 5 ml of the spray into each nostril.
